(19) United States
(12) Patent Application Publication
(10) Pub. No.: US 2014/0230313 A1
(43) Pub. Date: Aug. 21, 2014

(54) Sustained Release Delivery Devices

(71) Applicant: Massachusetts Institute of Technology, Cambridge, MA (US)

(72) Inventor: Noel M. Elman, Cambridge, MA (US)

(73) Assignee: Massachusetts Institute of Technology, Cambridge, MA (US)

(21) Appl. No.: 14/171,442

(22) Filed: Feb. 3, 2014

Related U.S. Application Data
(60) Provisional application No. 61/761,668, filed on Feb. 6, 2013.

(51) Int. Cl. A61L 9/03; A01M 25/00; A61L 9/12

(52) U.S. Cl. CPC A61L 9/032; A61L 9/12; A01M 25/00
USPC 43/124; 239/57; 392/386; 239/6

(57) ABSTRACT

Exemplary embodiments provide dispensers and delivery devices that release their contents at sustained rates. An exemplary device may include one or more reservoirs for holding a solid, semisolid, liquid or gaseous substance that may be released outside the device. Exemplary devices enable sustained release of their contents over a predetermined period of time, for example, up to a month or more. Exemplary devices also enable release of the contents according to, for example, predetermined release rates and profiles customized for the use of the device. Each reservoir of the device may include one or more apertures through which the contents may be released outside the reservoir. Each aperture may be covered by one or more porous membranes to allow sustained release of the contents of the reservoir.

### CROSS-REFERENCE TO RELATED APPLICATION(S)

This application relates to and claims priority from U.S. Provisional Patent Application No. 61/761,668 filed on Feb. 6, 2013, the entire disclosure of which is incorporated herein by reference in its entirety.

### GOVERNMENT SUPPORT

This invention was made with Government support under Grant awarded by the Department of the U.S. Army and the U.S. Army Research Office under Contract #W911QY-12-1-0005 and W911NF-13-D-0001. The Government has certain rights in this invention.

### SUMMARY

Exemplary embodiments provide dispensers and delivery devices that release their contents at sustained rates. Exemplary devices enable sustained release of their contents over a predetermined period of time, for example, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 days, and above.

In accordance with one exemplary embodiment, a delivery device is provided for sustained release of a substance. The delivery device includes a substrate, a reservoir formed in the substrate for holding the substance, and a porous membrane covering an aperture in the reservoir for sustained release of the substance through the pores of the membrane. Exemplary substances provided in the reservoir and released by a delivery device may include, but are not limited to, a pesticide, an insecticide, an insect repellant, a perfume, and the like. In an exemplary embodiment, the substrate of the delivery device may be biodegradable.

In an exemplary embodiment, the delivery device may include one or more sensors for detecting one or more environmental characteristics in which the delivery device is deployed. Exemplary environment characteristics may include, but are not limited to, visual presence/absence of one or more factors (using a camera), temperature, humidity, noise (using a microphone), wind, time of day, light conditions (e.g., whether it is night or day), the presence of a particular chemical or biological compound in the environment, and the like.

In an exemplary embodiment, the delivery device may include a propulsion mechanism for propelling the substance out of the reservoir to increase the rate at which the substance is released from the reservoir.

In an exemplary embodiment, the delivery device may include a heating mechanism for heating the substance in the reservoir.

In an exemplary embodiment, the delivery device may include one or more attachment mechanisms for attaching the delivery device to an external structure, for example, a garment (e.g., a jacket), a user's body (e.g., a user's wrist), a habitation structure (e.g., a tent), a vehicle (e.g., a tank), and the like.

In accordance with another exemplary embodiment, an array of delivery devices is provided for sustained release of one or more substances. Each delivery device in the array may include a substrate, a reservoir formed in the substrate for holding the substance, and a porous membrane covering an aperture in the reservoir for sustained release of the substance through the pores of the membrane. In one embodiment, all of the delivery devices in the array may contain and release the same substance. In another embodiment, at least two delivery devices in the array may contain and release different substances.

In an exemplary delivery device array, porous membranes of at least two or more of the delivery devices may be oriented in the same direction. In another exemplary delivery device array, porous membranes of at least two or more of the delivery devices may be oriented in different directions.

### BRIEF DESCRIPTION OF THE DRAWINGS

The foregoing and other objects, aspects, features, and advantages of exemplary embodiments will become more apparent and may be better understood by referring to the following description taken in conjunction with the accompanying drawings, in which:

FIG. 1A illustrates a block diagram of an exemplary delivery device.

FIG. 1B illustrates a side schematic view of an exemplary delivery device.

FIG. 1C illustrates a perspective view of an exemplary delivery device having a single reservoir.

FIG. 1D illustrates a perspective view of an exemplary delivery device having multiple reservoirs.

FIG. 1E illustrates a top down schematic view of a powered heating element.

FIG. 1F illustrates a top down schematic view of an exothermic heating element.

FIG. 1G illustrates a graph of temperature versus time showing the effect of an exothermic reaction on the substance in the reservoir.

FIG. 1H is a side cross-sectional view of an exemplary delivery device having exemplary connectors to connect the delivery device to a further delivery device.

FIG. 1I is a side cross-sectional view of two exemplary delivery devices stacked together.

FIG. 1J is a graph illustrating the diffusive effects of transfluthrin.

FIG. 1K is a graph illustrating the evaporation rate of different concentrations of transfluthrin and Deet.

FIG. 1L is an image of a pore opened using an exemplary fuse.

FIG. 1M is a set of graphs illustrating the opening of the pores using various combinations of short electric pulses.

FIG. 1N is a perspective view of an exemplary device having electric fuses for opening individual membranes.

FIG. 2 illustrates an exemplary disposable delivery device that may be formed of biodegradable materials.

FIG. 3 illustrates an exemplary schematic view of another embodiment of an exemplary delivery device.

FIG. 4A is a schematic representation of a concentration profile of a substance released by a conventional delivery device.

FIG. 4B is a schematic representation of a concentration profile of a substance released by an exemplary delivery device.

FIG. 5A schematically represents a uniform concentration gradient of a substance released from an exemplary delivery device.

FIG. 5B schematically represents concentration gradients of a substance released from an exemplary delivery device array including multiple delivery devices or reservoirs.

FIG. 6A is a schematic showing exemplary delivery devices provided on the body of a soldier.

FIG. 6B is a schematic showing exemplary delivery devices provided on a tent.

FIG. 6C is a schematic showing exemplary delivery devices provided on a vehicle.

FIG. 7 illustrates an exemplary delivery device provided on an item of clothing.

FIGS. 8A-8C illustrate an exemplary array of delivery devices.

FIG. 8D illustrates a perspective view of an exemplary wearable delivery device.

FIG. 8E illustrates a top-down view of an exemplary wearable delivery device.

FIG. 8F is an image of an exemplary wearable delivery device.

FIG. 9 illustrates a setup of a controlled experiment performed to determine the efficacy of an exemplary array of delivery devices in deterring pest bites.

FIG. 10 is a histogram of the percentage of mosquitoes that bit the test arm of FIG. 9 over 28 days of the experiment.

FIG. 11 is a histogram showing the percentage of mosquito bites of the test arm of

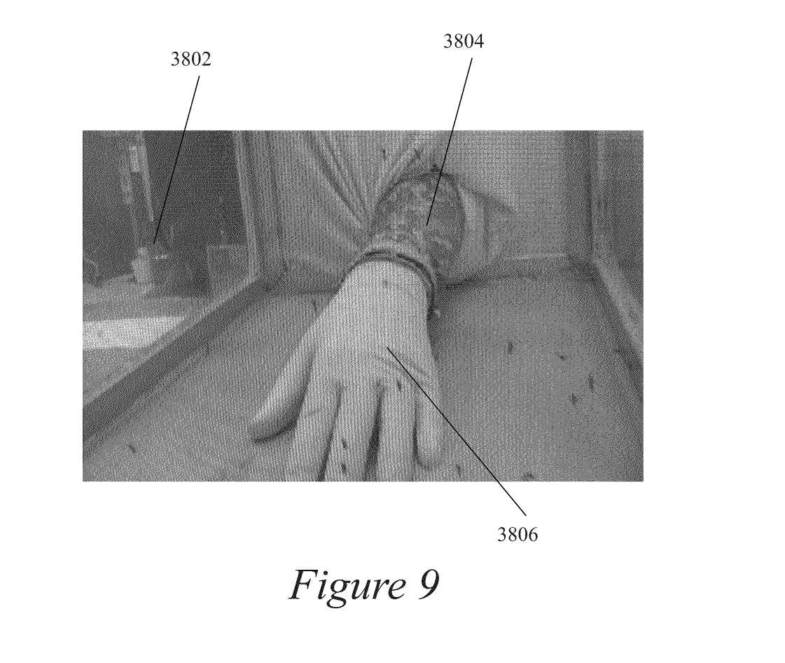

FIG. 9 along the y-axis and the number of days of the experiment along the x-axis.

FIGS. 12A and 12B illustrate volatility changes of N,N-Diethyl-meta-oluaiide (DEET) with temperature.

FIGS. 13A-13C illustrate volatility changes of transfluthrin with temperature.

### DETAILED DESCRIPTION

Exemplary embodiments provide dispensers and delivery devices that release their contents at sustained rates. An exemplary device may include one or more reservoirs for holding a solid, semisolid (e.g., gel), liquid or gaseous substance that may be released outside the device.

Exemplary devices enable sustained release of their contents over a predetermined period of time, for example, up to a month or more. Exemplary devices also enable release of the contents according to, for example, predetermined release rates and profiles customized for the use of the devices.

Each reservoir of the device may include one or more apertures through which the contents may be released outside the reservoir. Each aperture may be covered by one or more porous membranes to allow sustained release of the contents of the reservoir.

In some exemplary embodiments, a single reservoir may be provided. In other exemplary embodiments, a plurality of reservoirs may be provided. In an exemplary embodiment including multiple reservoirs, the reservoirs may be arranged in a two-dimensional or three-dimensional array. Reservoirs in an array may be selectively configured and positioned to maximize the coverage and efficacy of the substance delivered by the device and to minimize direct exposure of the user to the substance. An exemplary reservoir may hold any suitable volume of a substance, for example, ranging from about 1 cubic mm to about 100 cubic cm, but reservoirs are not limited to this exemplary range of volumes.

Certain exemplary dispensers are passive devices that do not rely on electro-mechanical release mechanisms to release the contents of the device. Exemplary passive devices include reservoirs that have structure, function and operation configured to achieve sustained release of the contents from the reservoirs. A porous membrane in a passive device may have a predetermined thickness and may include pores having a predetermined diameter and density in order to release the contents of the reservoir in a sustained manner and in a desired spatial profile.

Some exemplary delivery devices may be used in creating effective, protective low-toxicity plumes of substances such as pesticides, perfumes, and the like. Sustained release of a pesticide at a sustained rate may ensure adequate protection against pests, while ensuring that the toxicity of the pesticide does not exceed a maximum safe limit. Dispersal of safe pesticides using exemplary devices provides a method of prevention against several vector-borne illnesses, for example, malaria, dengue, and the like. Furthermore, the use of exemplary devices for the release of a pesticide advantageously avoids the need to apply the pesticide directly to the user's skin.

Exemplary miniaturized delivery devices may be seamlessly integrated with the skin and/or clothing of a user, and/or any portable infrastructure. Certain exemplary delivery devices may be implemented as wearable devices that may be affixed to a user. The wearable devices may also be affixed to or embedded in the clothing of a user. Certain exemplary delivery devices may be implemented as protection devices may be disposed along the perimeter or boundary of a desired space so that the contents of the device are released into the space. Certain exemplary delivery devices may be implemented as protection devices that may be affixed to temporary or permanent dwelling units, for example, rooms in a house or apartments, tents, and the like.

Multiple reservoirs or multiple delivery devices may be provided or connected in serial fashion or in parallel fashion for programmable operation of their release profiles. Reservoirs provided in serial fashion may be actuated to release one or more substances in sequence, for example, at different times. Reservoirs provided in parallel fashion may be actuated to release one or more substances concurrently.

### Definitions of Terms

As used herein, the term “substance” refers to any type of solid, semisolid (e.g., gel), liquid or gaseous material that may be held in an exemplary delivery device and released from the device into the environment. Exemplary substances may include, but are not limited to, pesticides, insecticides, insect repellants, perfumes, attractants, pheromones, and the like.

As used herein, the terms “delivery device,” “dispenser device” and “dispenser” refer to a micro-fabricated device that contains a substance and that releases the substance through a porous membrane. In some embodiments, an exemplary delivery device does not employ electrical energy or electro-mechanical release mechanisms to release the substance contained in the device.

As used herein, the term “porous membrane” refers to a standing structure including a plurality of pores or apparatuses that may be used to cap an opening in a reservoir in a delivery device or as an opening to a delivery device. The pores of an exemplary porous membrane are dimensioned and configured so that the membrane is permeable to the contents of the delivery device that are released outside the device through the porous membrane but may also act as a barrier to entry, for example, a moisture barrier. Exemplary materials that may be used to form a porous membrane include, but are not limited to, silicon, starch, polylactic acid (PLA), and the like. An exemplary porous membrane may be formed of a naturally permeable material (e.g., silicon), or may have pores engineered in the membrane.

### Exemplary Embodiments

Certain exemplary dispensing devices are passive devices that do not rely on electro-mechanical release mechanisms to release the contents of the device. Exemplary passive delivery devices include one or more reservoirs that have structure, function and operation configured to achieve sustained release of the contents from the reservoirs. In an exemplary embodiment, multiple reservoirs may take the form of an array of reservoirs. Each reservoir may include a porous membrane that is configured to release the contents in a sustained manner. Alternatively, in some embodiments, a porous membrane may be placed in one or more walls of the device in place of a cap on one or more reservoirs. The membrane can function as a barrier to moisture, for example, the membrane can facilitate the exit of the substance in the reservoir while preventing the entry of moisture (e.g., water) into the reservoir.

Each porous membrane may have a predetermined thickness and may include pores having a predetermined diameter and density in order to release the contents of the reservoir in a sustained manner. In an exemplary embodiment, a predetermined release rate or a predetermined range of release rates may be achieved by configuring the porous membrane to a predetermined thickness or a predetermined range of thicknesses. In another exemplary embodiment, a predetermined release rate or a predetermined range of release rates may be achieved by configuring the diameters of pores in the porous membrane to a predetermined diameter or a predetermined range of diameters. In another exemplary embodiment, a predetermined release rate or a predetermined range of release rates may be achieved by configuring the density of pores in the porous membrane to a predetermined density or a predetermined range of densities. In other exemplary embodiments, two or more of the above variables may be configured to achieve predetermined release rate or a predetermined range of release rates.

Exemplary passive dispensing devices are advantageous in their ease of use and low manufacture cost. The ease of use and low cost of the passive devices facilitate distribution of the devices in large numbers in different settings and scenarios to release the contents of the devices to cover large areas. The ease of use and lack of a need of maintenance allows the passive devices to be distributed without requirement activation before distribution, or maintenance or upkeep after distribution.

An exemplary porous membrane, provided in a delivery device opening, may include a plurality of pores to provide a porous surface. A substance stored in the reservoir may leave the delivery device through the pores in the porous membrane, for example, by diffusion. In some examples, the substance may be released continuously at a gradual release rate. A porous membrane in a passive device may have a predetermined thickness and may include pores having a predetermined diameter and density in order to release the contents of the reservoir in a sustained manner and in a desired spatial profile.

Permeability of the porous membrane to the substance in the reservoir allows the substance in the reservoir to be released outside the reservoir. The substance may be released from the reservoir by any number of mechanisms including, but not limited to, diffusion, osmosis, mechanical propulsion mechanisms, and the like. Release of the substance creates a plume outside the device.

Dimensions and cross-sectional geometry of the porous membrane including, but not limited to, the length, width and thickness, are configured to control the rate at which the substance is released from the reservoir. The rate of release of the substance may also be affected by the volatility of the substance.

Exemplary devices may be fabricated using any suitable mechanism including, but not limited to, injection molding, wafer fabrication, three dimensional printing, and the like.

Exemplary reservoirs and porous membranes that may be used in passive delivery devices are described in relation to exemplary reservoirs and porous membranes usable in active delivery devices.

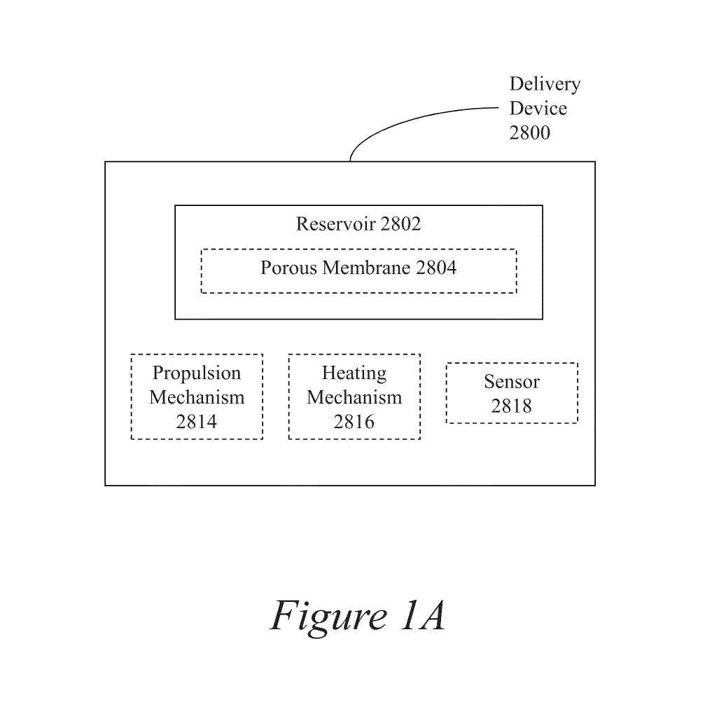

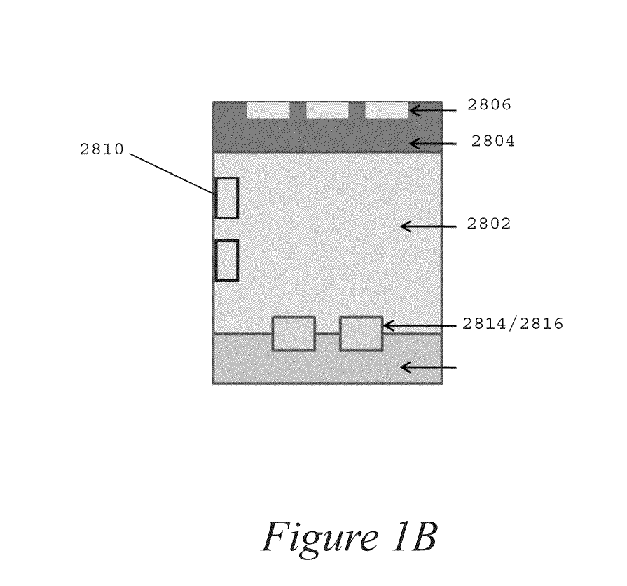

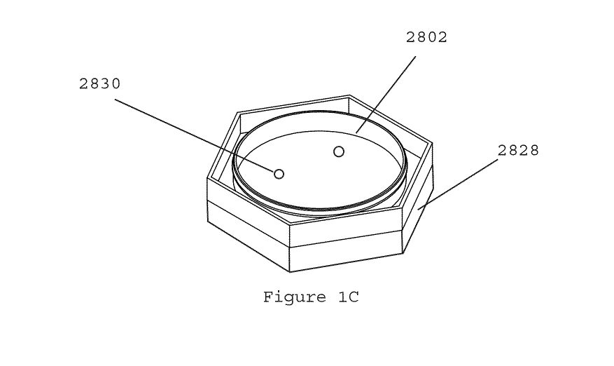

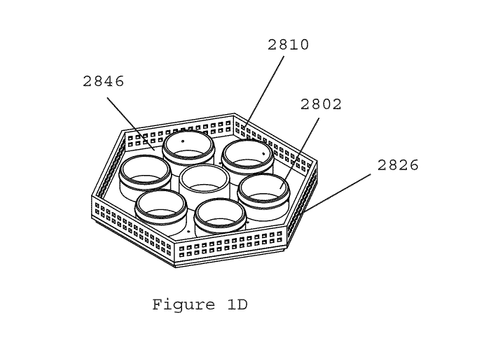

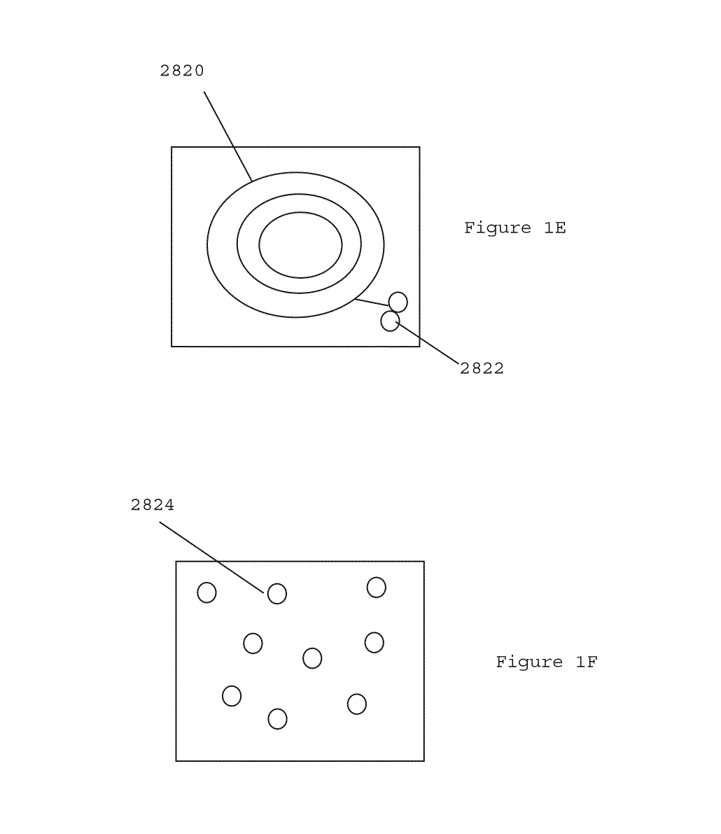

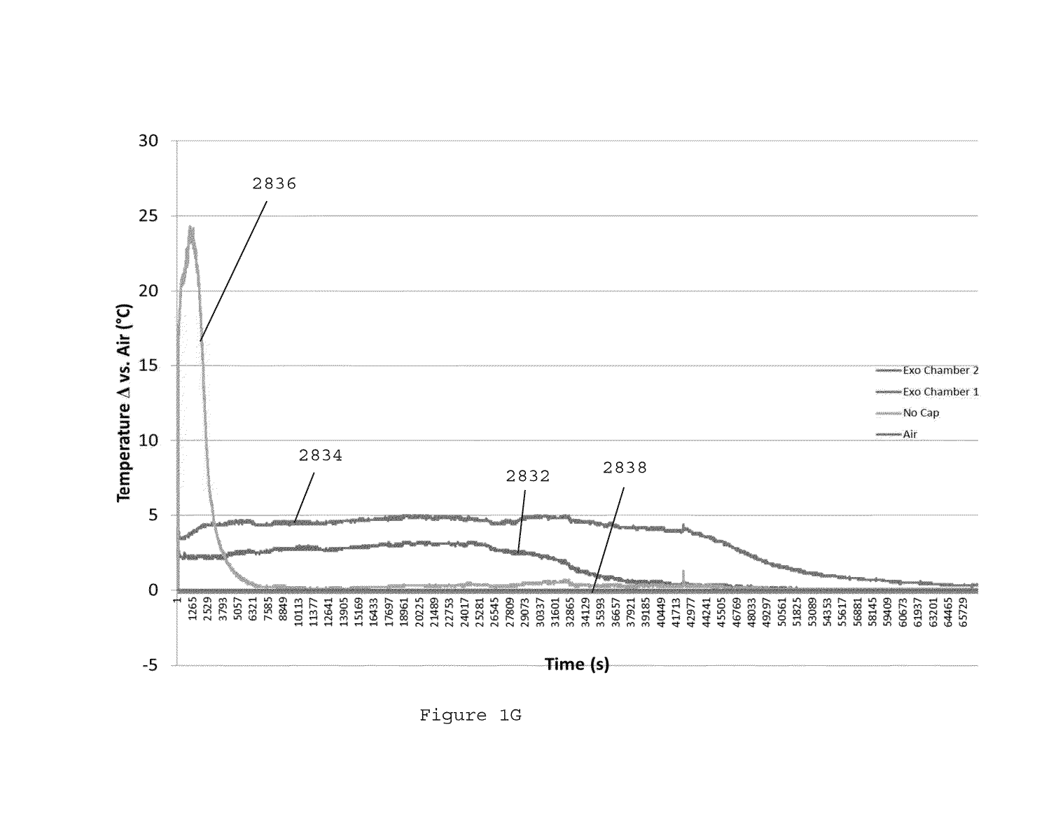

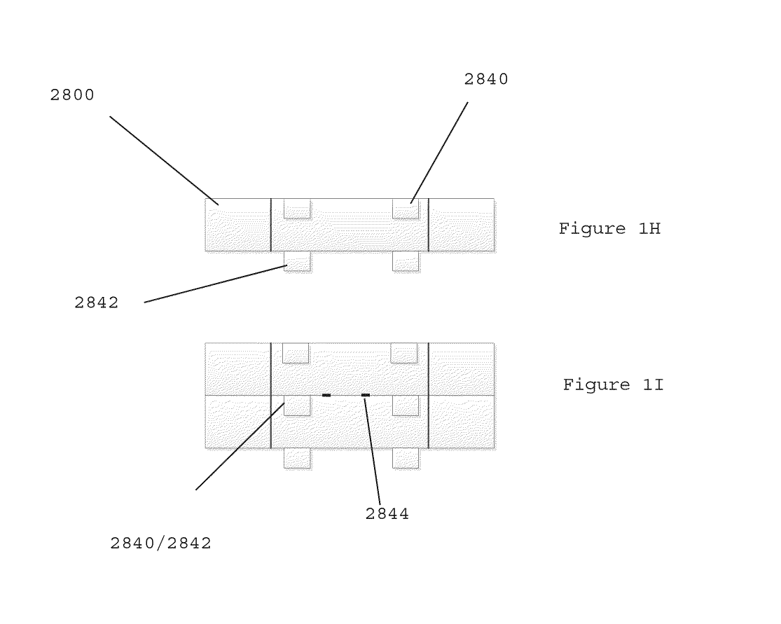

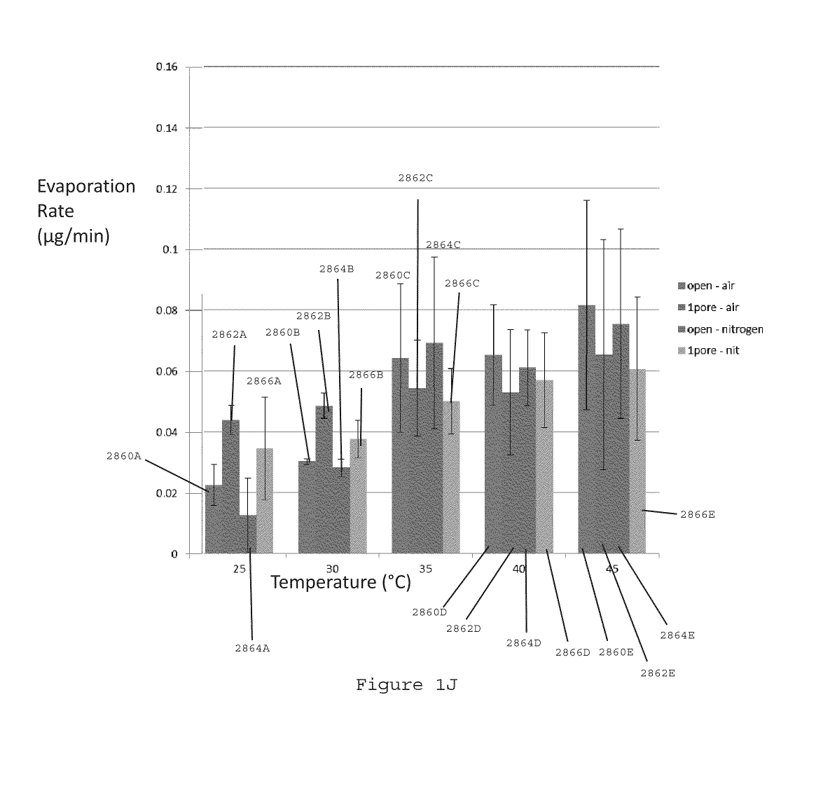

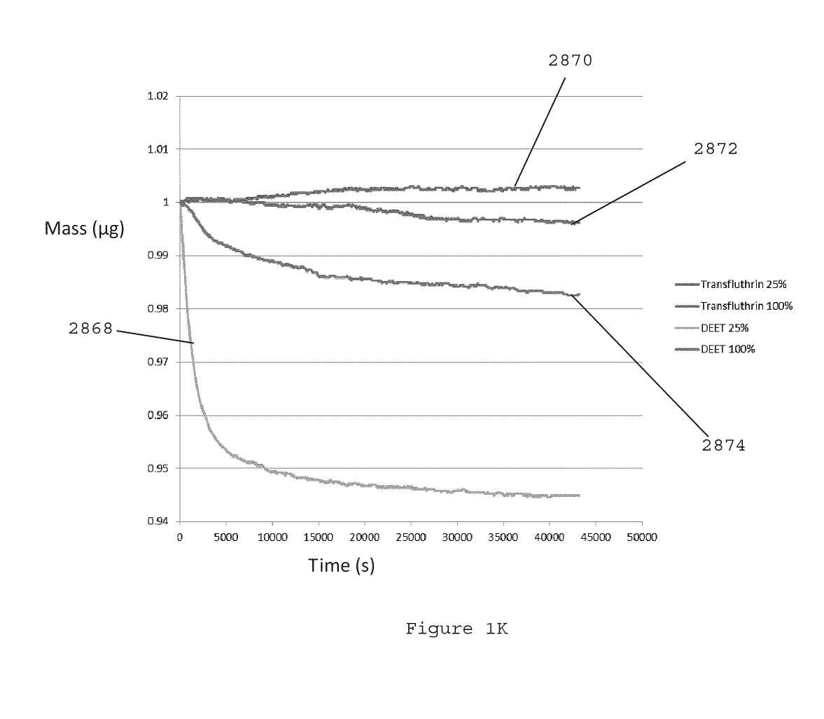

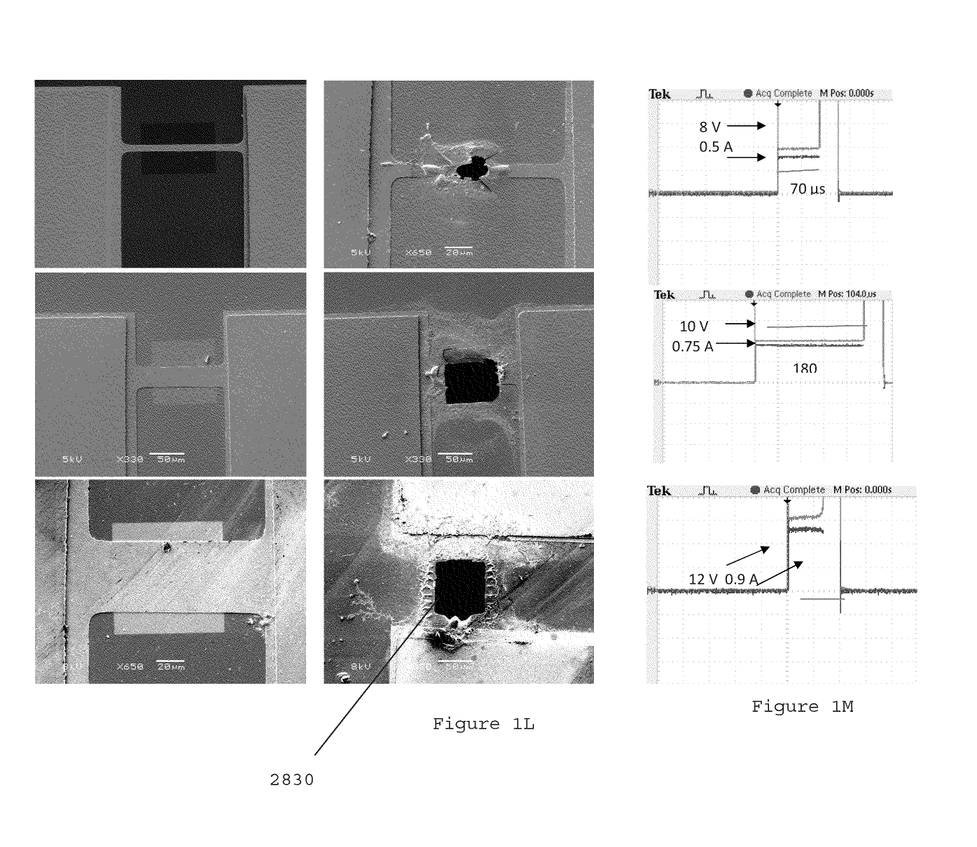

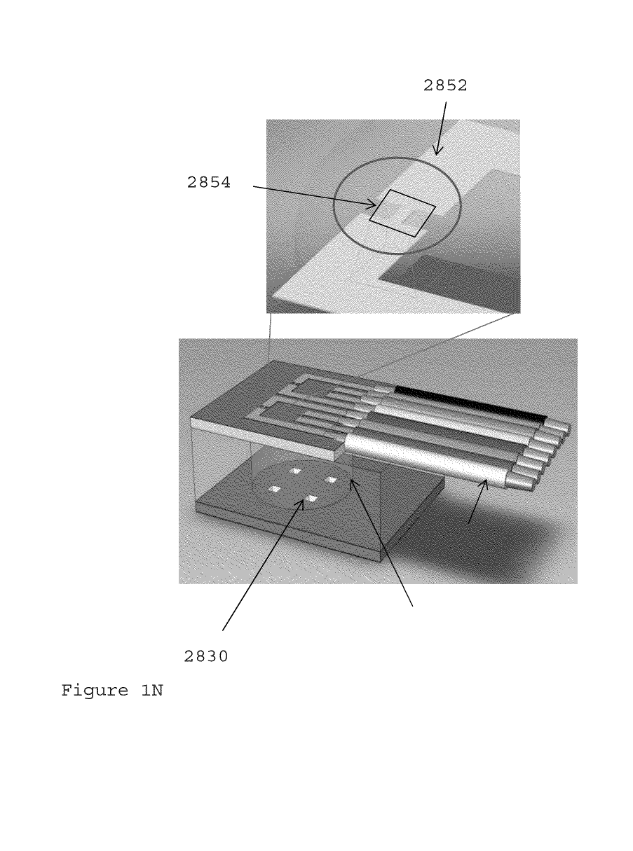

FIGS. 1A-1D are diagrams of an exemplary delivery device 2800. One of ordinary skill in the art will appreciate that the present invention is not limited to the specific exemplary embodiments described in connection with FIGS. 1A-1D. Many alterations and modifications may be made to the exemplary delivery device by those having ordinary skill in the art without departing from the spirit and scope of the invention. For example, delivery device 2800 is shown as being hexagonal in shape (see e.g., FIG. 1C), however, as one skilled in the art will understand, various other shapes can be utilized including, but not limited to, circular, square, rectangular, triangular, pentagonal etc.

An exemplary delivery device can include one or more reservoirs 2802 for holding one or more substances. For example, exemplary device 2800 can include a single reservoir that can be sized to encompass most of the delivery device (e.g., FIG. 1C). Alternatively, the exemplary delivery device can include a plurality of smaller reservoirs (e.g., FIG. 1D). Exemplary reservoirs may be formed of any suitable material including, but not limited to, plastic, metal, and the like. In some examples, an array of multiple micro-reservoirs may be formed using any suitable technique including, but not limited to, 3D printing, stereolithography techniques, high-precision plastic molding, and the like.

An exemplary reservoir may be formed in a substrate of the delivery device, and may include one or more open portions through which the content of the reservoir may be exposed or released into the treatment site. In some exemplary embodiments, a reservoir holds one or more sensors configured to sense a characteristic of the treatment site, for example, the temperature of the treatment site, the presence of a particular chemical or biological compound in the treatment site, and the like. In other exemplary embodiments, a reservoir holds one or more substances.

Exemplary delivery devices include one or more porous covers or membranes 2804 suspended over the open portions of each reservoir or forming a portion of one or more walls of the device. An exemplary porous membrane, provided in a reservoir opening, may include a plurality of pores 2806 to provide a porous surface. Pores can be sufficiently sized to facilitate escape of the substance from inside of the reservoir while preventing water from entering into the reservoir through pores 2806, and interacting with the substance in the reservoir. In some examples, each of the one or more reservoirs 2802 can have a single pore associated with the reservoir. In other examples, a plurality of pores can be associated with each reservoir. The exemplary delivery device can also have one or more side pores 2810, which can encompass all, or a portion, of each side 2826. Pores 2810 can be in addition to (e.g., the exemplary device has pores 2806 and side pores 2810), or in replacement of pores 2806 (e.g., the exemplary device only has side pores 2810).

In embodiments that include a membrane cover over the reservoir, the substance stored in the reservoir may leave the reservoir through the pores in the porous membrane, for example, by diffusion. In embodiments that do not include a membrane cover over the reservoir, the substance may be released continuously at a gradual release rate. A porous membrane in a passive device may have a predetermined thickness and may include pores having a predetermined diameter and density in order to release the contents of the reservoir in a sustained manner and in a desired spatial profile.

In one embodiment, a delivery device may include a propulsion mechanism 2814 that mechanically propels the substance out of the reservoir. This increases the rate at which the substance is released outside the reservoir to accommodate, for example, substances that must be delivered at a fast rate. In an exemplary embodiment, the propulsion mechanism may trigger bubble nucleation to rapidly eject a liquid substance out of the reservoir, for example, in a jet. The activation mechanism of the delivery device may thus be configured to achieve a desired release rate, for example, a slow rate that uses diffusion, a fast rate that uses an exemplary propulsion mechanism, a combination of fast and slow rates, a constant rate, a variable rate, and the like.

In one embodiment, a delivery device may include a heating mechanism 2816 including one or more heating elements associated with the reservoirs of the device to increase the volatility of the contents of the reservoirs. Exemplary heating elements can include an active or powered heating mechanism 2814 (e.g., FIG. 1E). For example, one or more heating elements or heating coils 2820 can be placed under or next to the substance. The heating element can be powered by one or more power elements (e.g., batteries 2822). The number of heating elements and the number of batteries can depend on the desired heat output, which can affect the release rate of the substance.

A further exemplary heating mechanism 2814 can include one or more chemicals or substances used to generate an exothermic reaction (e.g., exothermic reaction substance 2824 of FIG. 1F). For example, iron can be oxidized into iron oxide, and generate heat to affect the volatility and release rate of the liquid substance. Other exemplary substances that can be used for the exothermic reaction can include a combination of CaCO₃ (calcium carbonate) and CaO (calcium oxide or lime) to cause an exothermic reaction controlled by water. Other suitable chemicals can also be utilized that can generate an exothermic reaction. An advantage of such a reaction is that the reaction rate can be dependent on water, which can depend on humidity, which is beneficial under certain circumstances (e.g., for certain pesticides that are hydroscopic) as the overall volatility can also be affected (e.g., decreased) by humidity. As shown in FIG. 1C, exothermic reaction 2824 can take place in a chamber (e.g., exothermic reaction chamber 2828, which can be located below reservoir 2802). One or more exothermic reaction pores 2830 can be located between exothermic reaction chamber 2828 and reservoir 2802. An exemplary exothermic reaction pore size can be 1 mm, although larger and smaller pore sizes can be used. As shown in FIG. 1C, the exothermic reaction can take place in a reaction chamber located below the reservoir. Alternatively, or in addition to, the exothermic reaction can take place in the open area surrounding reservoir 2802 (e.g., open area 2846 of FIG. 1D). The length of time of the exothermic reaction can be a function of the pore size. For example, FIG. 1G illustrates a graph of temperature versus time with Exo Chamber 1 (element 2832) including 6 pores each 1 mm in size, Exo Chamber 2 (element 2834) including 6 pores each 2 mm in size, No CAP (element 2836) including 0 pores, and Air (2838) used as a reference. As is evident from the graph, the number of pores, and pore size can affect the temperature of the substance in the reservoir. An exemplary pore size can be 500 μm, although larger and smaller pore sizes can be used.

Increased volatility of the contents increases the overall flux of the contents out of the reservoirs. The activation mechanism of the delivery device may thus be configured to achieve a desired release rate, for example, a slow rate that uses diffusion, a fast rate that uses an exemplary heating mechanism, a combination of fast and slow rates, a constant rate, a variable rate, and the like. A thermo-gravimetric analysis can be used to determine the surface area and diffusive effects of a particular substance. For example, FIG. 1J, illustrates such effects using transfluthrin as an exemplary substance. As shown in FIG. 1J, the evaporation rate can vary depending on the whether the container is completely open and the substance is released into air (elements 2860A-E), whether the container is covered by a membrane having one pore with the substance being released into the air (elements 2862A-E), whether the container is completely open and the substance is released into nitrogen (elements 2864A-E), and whether the container is covered by a membrane having one pore with the substance being released into nitrogen (elements 2866A-E). Additionally, the release rate can be dependent on the concentration of the substance. For example, as shown in FIG. 1K, which illustrates the effect on the release rates of 25% transfluthrin and 75% alcohol (element 2874), 100% transfluthrin (element 2870), 25% deet and 75% alcohol (element 2868) and 100% deet (element 2872).

The exemplary device may be configured to achieve a desired release rate taking into account the volatility changes due to temperature. Volatility may depend on the temperature. For example, as shown in FIGS. 12A and 12B, varying the temperature can affect the evaporation rate of deet. FIGS. 13A-13C illustrate the effect of varying the temperature on transfluthrin.

The exemplary delivery device can include one or more structural components configured to cover or close each pore 2830 until a desired circumstance is achieved (e.g., a specific temperature of the substance or a specific time period). For example, as shown in FIGS. 1L and 1N, actuators can be used to open the reservoir 2802 through the use of electric fuses 2852 opening an individual membrane 2854 upon application of short electric pulses (e.g., as shown in FIG. 1M).

The delivery device may include one or more sensors 2818 configured to sense a characteristic of the environment, for example, temperature, humidity, noise (e.g., using one or more microphones), wind, presence/absence of visual cues (e.g., using one or more cameras), the presence of a particular chemical or biological compound, and the like.

The delivery device can include various mechanisms or arrangements to stack to other delivery devices, facilitating easier storage of multiple delivery devices, or to increase the potency or duration of the delivery device. For example, as shown in FIG. 1H, delivery device 2800 can include one or more protrusions 2842 to connect to another delivery device. Delivery device 2800 can also include one or more openings 2840 that protrusions 2842 can fit in and stack, for example, using an interference fit or other suitable connection mechanism. FIG. 1I illustrates two delivery devices stacked together. As shown, the protrusions 2842 of one delivery device can fit into the openings 2840 of a second delivery device, joining the two delivery devices together as shown by element number 2840/2842. The delivery devices can include one or more pores 2844 that can facilitate the transfer of the substance of one delivery device through the other delivery device when a longer duration, or more potent delivery, is desired.

Certain exemplary passive dispensing devices are advantageous in that the devices are simple in construction and may be formed of biodegradable materials and may be allowed to biodegrade in a sustained fashion and be bio-absorbed by the environment without leaving any inorganic residue in the area of deployment. Exemplary biodegradable materials that may be used to form components of a recyclable device include, but are not limited to, polylactic-co-glycolic acid (PLGA) of various dimensions and crosslinks, polylactic acid (PLA) of various dimensions and crosslinks, and the like. Exemplary passive dispensing devices may also be formed of non-biodegradable materials including, but not limited to, polymeric materials. These exemplary devices are fully recyclable and do not leave residues or signatures in the environment. In exemplary devices, the release profile of a substance may be controlled temporally through changes in the molecular weight of the porous membrane.

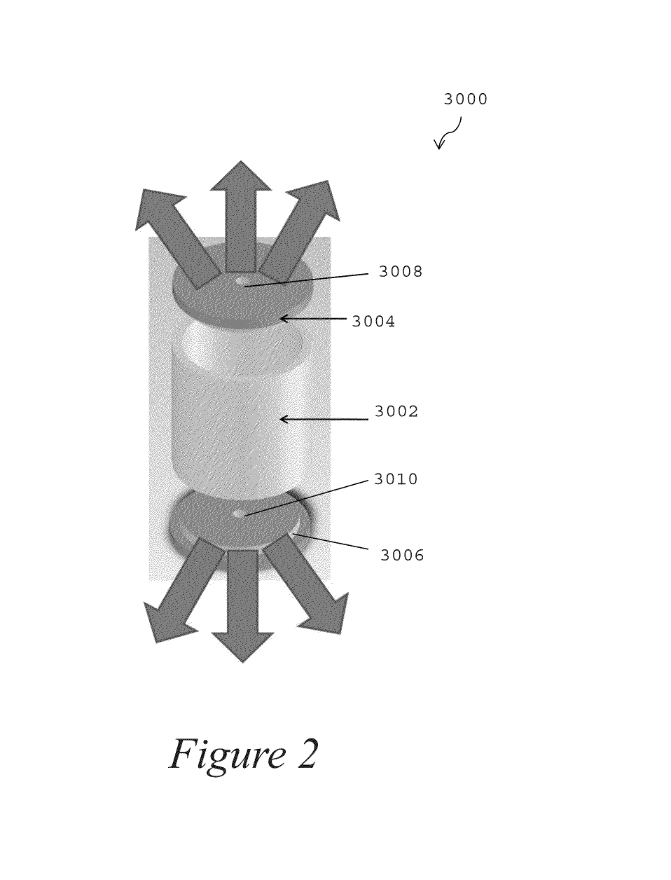

FIG. 2 illustrates an exemplary disposable passive delivery device 3000 that may be formed of biodegradable materials. In device 3000, a biodegradable reservoir 3002 is provided that includes one or more porous membranes 3004, 3006 that are also biodegradable. Each porous membrane 3004, 3006 may include one or more orifices or pores 3008, 3010 that enable a substance to be released from the reservoir 3002. The thickness of the porous membranes 3004, 3006 and/or the dimensions of the orifices 3008, 3010 may be configured to control the release rate of the substance and act as a moisture barrier to prevent entrance of moisture.

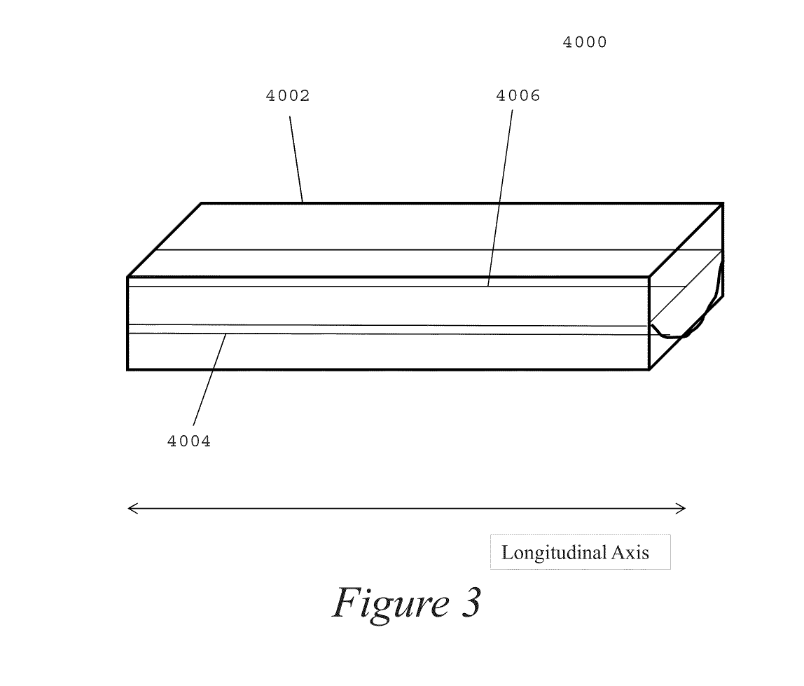

FIG. 3 illustrates an exemplary schematic view of another embodiment 4000 of an exemplary delivery device. Device 4000 includes a substantially box-shaped outer container 4002, although other suitable shapes may also be used. Within the outer container 4002, an inner container 4004 is provided in a hanging manner. The inner container 4004 may include a releasable substance, and may include pores in one or more of its surfaces for release of the substance from the inner container 4004. Similarly, the outer container 4002 may include one or more pores so that the substance (after release from the inner container 4004) is released outside the outer container 4002.

The inner container 4004 may be shaped as a half-cylinder split along its longitudinal axis in some embodiments, in which the cross-sectional face is that of a semicircle or semi-oval and in which the longitudinal axis of the container extends along the length of the cylindrical shape. The inner container 4004 is provided within the outer container 4002 such that it freely hangs and pivots about its central longitudinal axis 4006 (along its largest flat surface) and so that its largest flat surface along the longitudinal axis always faces upward relative to the outer container 4002. This may be implemented by coupling the inner container 4004 only at the ends of its central longitudinal axis 4006 to the outer container 4002. This enables the inner container 4004 to pivot about its central longitudinal axis 4006 so that the largest flat surface of the container 4004 faces upward within the device. This implementation enables the device 4000 to be deployed in any manner without concern about the eventual orientation of the inner container 4004. For example, the device may be deployed by hand drop and will achieve the desired upward facing orientation of the inner container 4004 when the device reaches the ground. This advantageously prevents the substance from leaking out of the device that would otherwise occur in the inner container landed in an upside-down orientation.

### Exemplification and Use

Exemplary embodiments, in contrast, enable control over the spatial distribution of a substance in time, that is, both spatial and time resolutions. As such, exemplary delivery devices may be used in providing tailored concentration profiles of a substance and may be designed to achieve sustained release of a substance over weeks with minimal exposure to humans. The combination of the release kinetics profiles with spatial arraying of the reservoirs and/or delivery devices result in concentration peaks of a substance in a specific spatial direction (e.g., in a direction projecting from a porous membrane orifice) for a specific period of time. That is, the selective activation of a plurality of reservoirs and/or a plurality of delivery devices enables control over the concentration peak of a substance, the specific direction(s) of release, and the times and time durations of release.

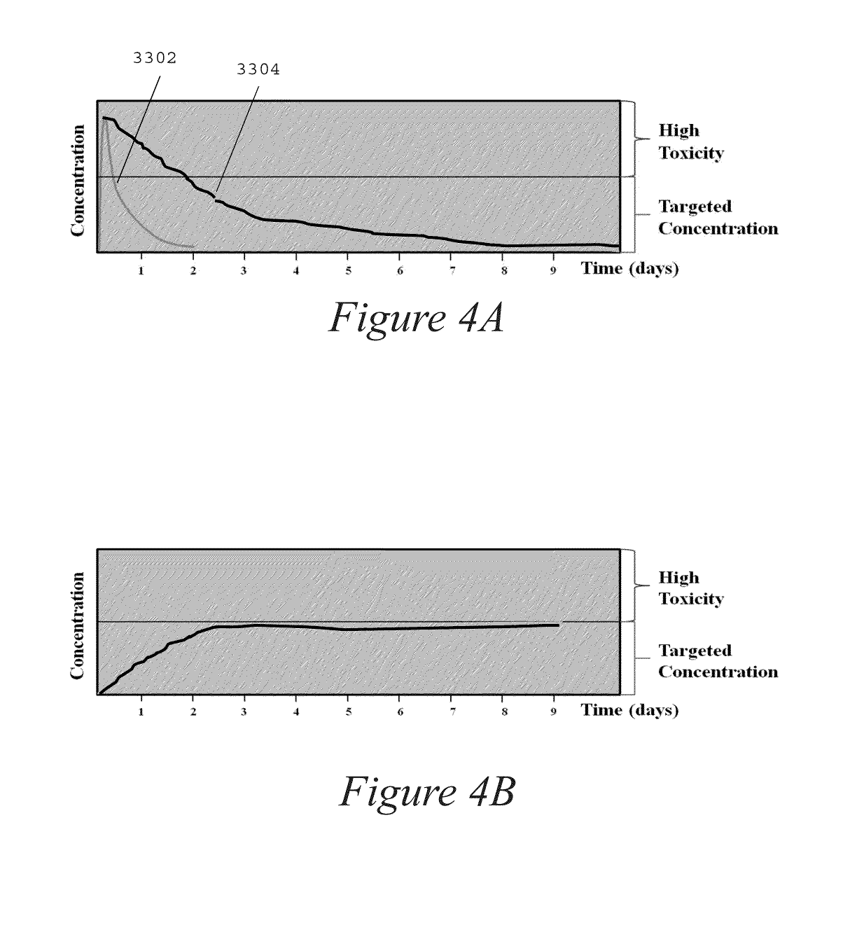

In contrast, conventional device delivery devices are unable to provide controlled concentration profiles of substances released. FIG. 4A is a schematic representation of the concentration profile of a substance released by a conventional delivery device or technique. As depicted by release profile 3302, topical spraying of a substance (e.g., a pest deterrent) results in a sudden increase in its concentration in the environment, overshooting to a high toxicity range and decaying rapidly, within a few hours, below the targeted concentration level or range of the substance. As depicted by release profile 3304, chemically treated fabric exhibit continuous release of a substance (e.g., a pesticide) by means of desorption or uncontrolled volatility. The release profile typically starts at a higher concentration in the high toxicity range that gradually decays within two days to a less than the targeted concentration level or range. Therefore, neither conventional technique provides sustained protection within the targeted safety concentration level or range.

The release profiles of substances may be tailored by exemplary delivery devices to achieve a targeted pesticide concentration level or range within safety limits. FIG. 4B is a schematic representation of the concentration profile of a substance released by an exemplary passive delivery device. An essentially constant concentration profile may be achieved by an exemplary passive delivery device. The material of the device and the structural design of the reservoirs may be configured to provide a sustained release of a substance. In addition, the porous membrane of the exemplary delivery device may be configured to achieve a desired concentration level or range by reducing the time required for reaching steady-state release kinetics. Other parameters that may be configured to control the concentration profile include, but are not limited to, pore size, pore density, membrane thickness, as well as diffusivity and volatility of the chemical compounds, and the like.

Exemplary delivery devices may be used to achieve desired spatial concentration gradients by selective arraying of reservoirs. In an exemplary array of delivery devices, porous membranes of at least two or more of the delivery devices may be oriented in the same direction. In another exemplary array of delivery devices, porous membranes of at least two or more of the delivery devices may be oriented in different directions.

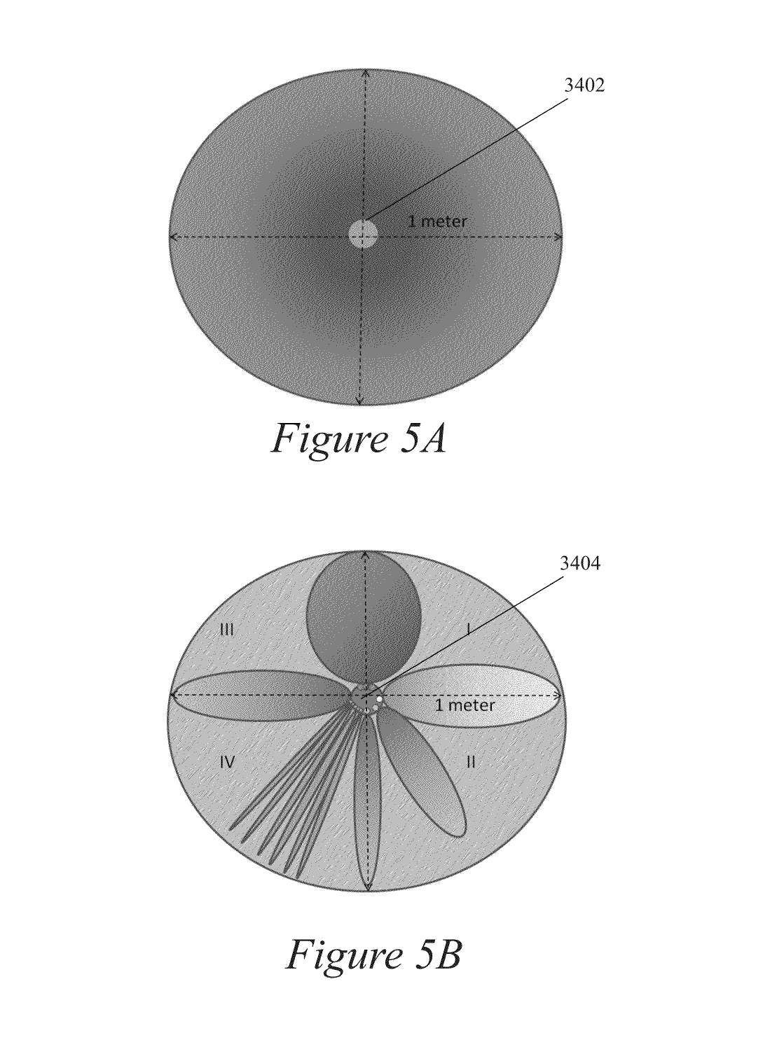

FIG. 5A schematically represents an essentially uniform concentration gradient of a substance released from a single source delivery device 3402. The concentration gradient is a uniformly decaying distribution over space, which is difficult to achieve in conventional techniques due to environmental conditions, e.g., humidity, wind speed. Exemplary delivery device arrays enable spatial targeting as well as selective distribution of substance concentration. FIG. 5B schematically represents concentration gradients of a substance released in three dimensions according to a desired spatial target from an exemplary delivery device array including multiple delivery devices or reservoirs 3404. That is, the concentration levels and gradients in space may be adjusted by defining different release domains at various concentrations. The devices in an exemplary array may be directed or configured to release a substance in the same direction or in different directions. For example, the devices may be located at a location, and may be directed or configured to release a substance at different angles in a circle centered at the location. In an example, the substance may be released to cover a substantially circular area centered at the location. In other examples, the substance may be released to cover regions at about 0, 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360 degrees, and, all intermediate angles, around the central location at which the devices are located. An advantage of this technique is in targeting exposed areas, e.g., tent entrances.

Some exemplary delivery devices may be used in creating effective, protective low-toxicity plumes of substances, such as pesticides. Exemplary delivery devices may be used to have a significant impact on disease prevention and eradication. Sustained release of a pesticide at a desired rate may provide adequate protection against pests, while limiting chemical exposure to the user and ensuring that the toxicity of the pesticide does not exceed a maximum safe limit. Dispersal of safe pesticides using exemplary devices provides a method of prevention against several vector-borne illnesses, for example, malaria, dengue, and the like. Furthermore, the use of exemplary devices for the release of a pesticide advantageously avoids the need to apply the pesticide or repellant directly to the user's skin. Exemplary delivery devices may be deployed, for example, in high-risk areas characterized by poor hygienic conditions, in disaster zones, and in areas affected by pest species that transmit debilitating and deadly diseases.

Conventional methods of dispersing pesticides are not very effective in controlling the population of adult pests in local areas. This deficiency is particularly prominent in efforts to control mosquitoes and flies, particularly those that transmit malaria and Leishmania. Some conventional methods of providing personal protection against pests involve treating clothing and skin with the pesticides. However, treated clothing constructed with open weaves (e.g., synthetics) allows pest bites through the clothing due to the porous material. In addition, volatile pesticides tend to evaporate from the treated clothing over time, and prevent the treated clothing from providing sustained protection. Other conventional methods include periodic spraying, direct deposition and topical application that provide large quantities of continual chemical exposure with potentially hazardous effects. That is, conventional methods of providing secondary protection are not effective at providing sustained long-term protection against pests with low toxicity exposure to users.

Certain conventional techniques of delivering pesticides involve spraying or chemically treating fabrics, for example, at the yarn or fabric level. A disadvantage with this conventional technique is the limitation on control over physical parameters that affect release of the pesticides, for example, the desired targeted concentration, toxicity profile, spatial directionality, coverage, and the like. Targeted structural areas in which a pesticide is dispersed by the conventional techniques may suffer overshooting, as high concentration levels are released above a safe region to eventually reach a targeted concentration that exponentially decreases as a function of time and space. For example, spraying of pesticides over tents or makeshift structures is typically characterized by a concentration peak. Similarly, targeted structural areas in which a pesticide is dispersed by the conventional techniques may suffer undershooting, in which the concentration of the pesticide is insufficiently high to provide protection for most of the treatment time. This is often the case with fabrics made of yarns that are chemically treated to continuously release a pesticide. As a result, the treated fabrics operate below the targeted concentration of the pesticide. Thus, conventional dispersal techniques are disadvantageous not only for continuous uncontrolled chemical exposure to humans, but also for their limited control in space and time.

Exemplary delivery devices enable personal protection by dispersing pesticides as a secondary means of reducing pest bites and reducing disease risk. Exemplary delivery devices may, in some exemplary embodiments, be attached to or associated with the skin or clothing of a user, and may gradually release a potent and volatile pesticide that provides long-term sustained protection to the user from pest bites. In addition, the release of the pesticide covers an area around the user to create a protective envelope that deters or kills the pests prior to landing on the user. Exemplary delivery devices may, in some exemplary embodiments, be attached to or be associated with a habitation of one or more users, for example, a tent, a camp, netting around a tent or bed, and the like. A plurality of exemplary delivery devices may be provided around a habitation, and may release pesticides at predefined times and at predefined rates to provide a pesticide barrier to pests attempting to enter the habitation.

Exemplary passive and active delivery devices may be used to hold and release any suitable substance. Exemplary pesticides may include those to combat diseases and/or pathogens borne by mosquitoes (e.g., yellow fever, malaria, and dengue), riatomine bug (e.g., chagas), and sand flies (e.g., leishmaniasis). Certain exemplary pesticides that may be released using exemplary passive and active delivery devices are listed in Table 1.

TABLE 1
List of exemplary pesticides

| Type of | |
|---|---|
| Pesticide | Name of Pesticide |
| Organochloride | Aldrin, Chlordane, Chlordecone, DDT, Dieldrin, Endosulfan, Endrin, |
| insecticides | Heptachlor, Hexachlorobenzene, Lindane (gamma- |
| | hexachlorocyclohexane), Methoxychlor, Mirex, Pentachlorophenol, |
| | TDE. |
| Organophosphate | Acephate, Azinphos-methyl, Bensulide, Chlorethoxyfos, Chlorpyrifos, |
| | Chlorpyriphos-methyl, Diazinon, Dichlorvos (DDVP), Dicrotophos, |
| | Dimethoate, Disulfoton, Ethoprop, Fenamiphos, Fenitrothion, Fenthion, |
| | Fosthiazate, Malathion, Methamidophos, Methidathion, Mevinphos, |
| | Monocrotophos, Naled, Omethoate, Oxydemeton-methyl, Parathion, |
| | Parathion-methyl, Phorate, Phosalone, Phosmet, Phostebupirim, Phoxim, |
| | Pirimiphos-methyl, Profenofos, Terbufos, Tetrachlorvinphos, Tribufos, |
| | Trichlorfon. |
| Carbamates | Aldicarb, Bendiocarb, Carbofuran, Carbaryl, Dioxacarb, Fenobucarb, |
| | Fenoxycarb, Isoprocarb, Methomyl, 2-(1-Methylpropyl)phenyl |
| | methylcarbamate. |
| Pyrethroids | Allethrin, Bifenthrin, Cyhalothrin, Lambda-cyhalothrin, Cypermethrin, |
| | Cyfluthrin, Deltamethrin, Etofenprox, Fenvalerate, Permethrin, |
| | Phenothrin, Prallethrin, Resmethrin, Tetramethrin, Tralomethrin, |
| | Transfluthrin. |
| Neonicotinoids | Acetamiprid, Clothianidin, Imidacloprid, Nitenpyram, Nithiazine, |
| | Thiacloprid, Thiamethoxam, Anabasine, Anethole, Annoninm Asimina |
| | for lice, Azadirachtin, Caffeine, Carapa, Cinnamaldehyde, Cinnamon |
| | leaf oil, Cinnamyl acetate, Deguelin, Derris, Desmodium caudatum, |
| | Eugenol, Linalool, Myristicin, Neem (Azadirachtin), Nicotiana rustica |
| | (nicotine), Peganum harmala, seeds (smoke from), root, Oregano oil, |
| | Polyketide, Pyrethrum, Quassia, Tetranortriterpenoid, Thymol. |

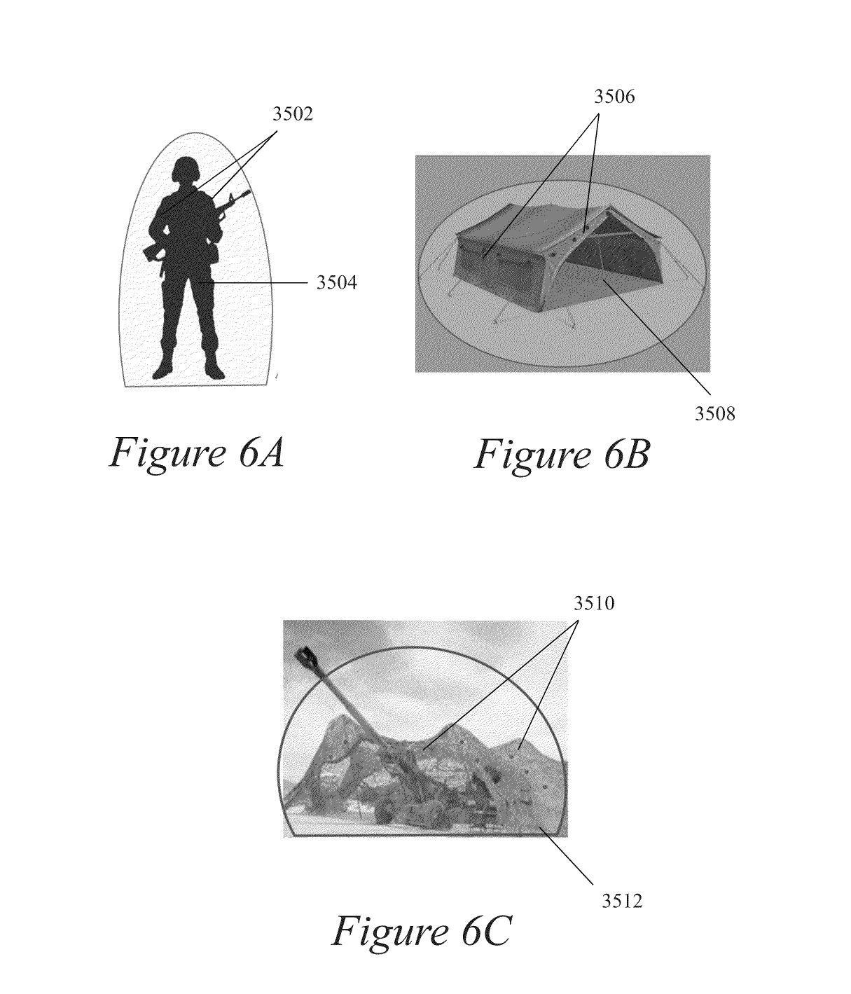

Exemplary embodiments may be associated with, at or on any suitable component. FIG. 6A is a schematic showing exemplary delivery devices 3502 provided on the body of a soldier 3504; FIG. 6B is a schematic showing exemplary delivery devices 3506 provided on a tent 3508; and FIG. 6C is a schematic showing exemplary delivery devices 3510 provided on netting 3512 covering a towable vehicle 3512.

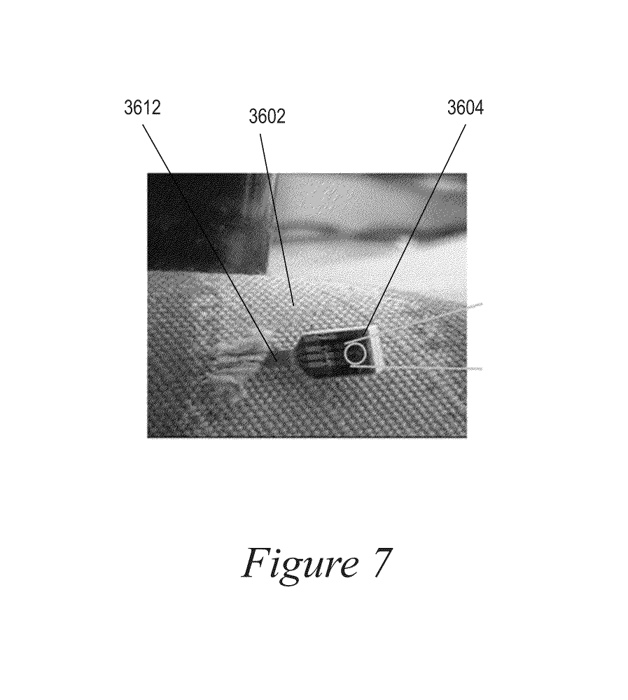

FIG. 7 illustrates a perspective view of a single delivery device 3604 embedded in or attached to an item of clothing 3602, for example, a jacket. The device 3604 includes a reservoir capped by a porous membrane. The device 3604 also includes an attachment mechanism 3612 that allows attachment of the device to the item of clothing 3602. Any suitable attachment mechanism may be used including, but not limited to, clips, threads, screws, hooks, and the like.

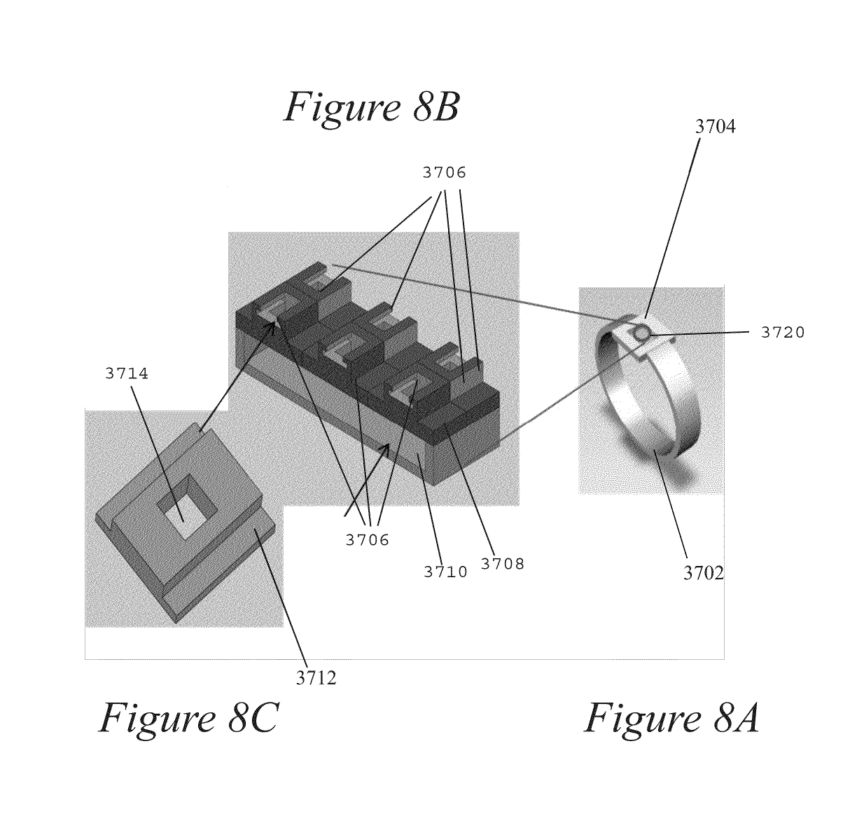

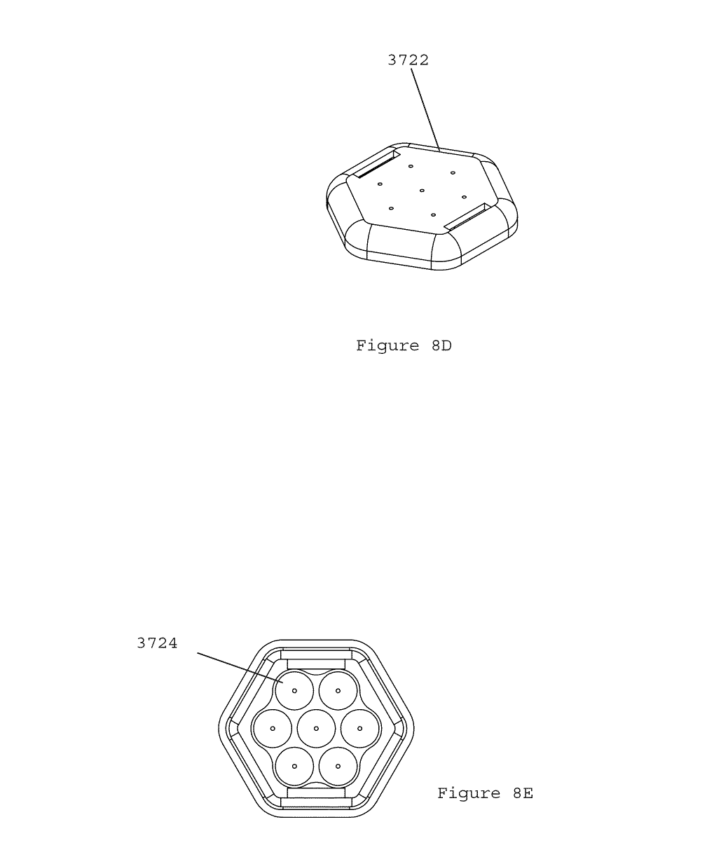

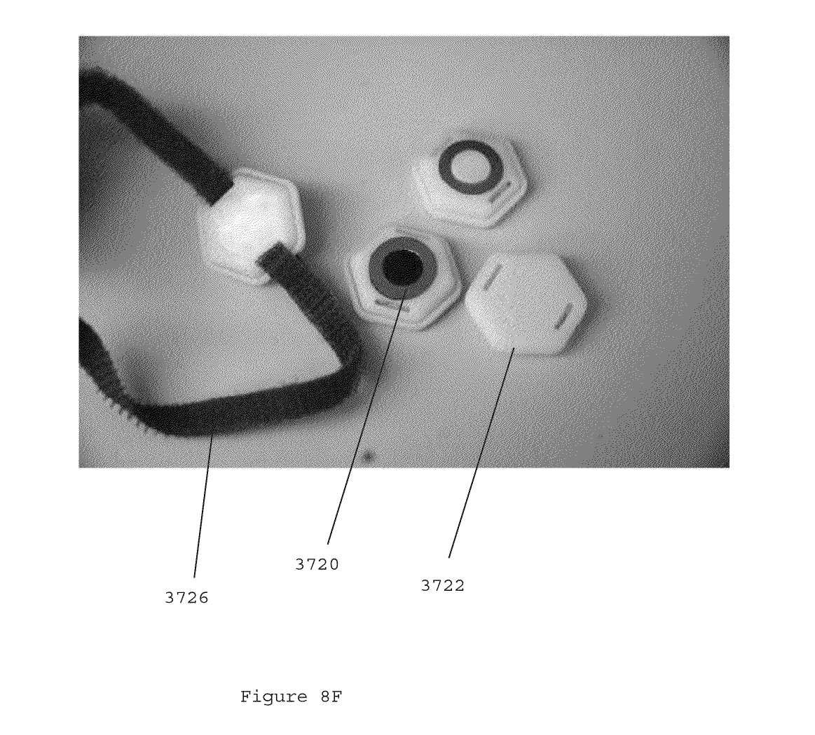

FIGS. 8A-8C illustrate an exemplary array of delivery devices provided on an item of clothing that may be used to more substances. FIG. 8A illustrates a perspective view of an item of clothing 3702 on which the array 3704 may be removably attached. In this case, the item of clothing 3702 is a wristband or armband. Clothing 3702 can include one or more heat transfer elements 3720, which can be composed of a metallic material or other suitable material. Heat transfer element 3720 can facilitate a transfer of body heat from the wearer of the clothing to the array 3704, to heat the substance in array 3704. FIG. 8B illustrates a perspective view of the array 3704 of delivery devices 3706 in which the devices are positioned on a base layer 3708. The base layer 3708 may be supported over an attachment mechanism 3710 that allows attachment of the array 3704 to the item of clothing 3702. Any suitable attachment mechanism may be used including, but not limited to, clips, threads, screws, hooks, and the like. Each delivery device includes a reservoir and a porous membrane for covering one or more surfaces of the reservoir. FIG. 8C illustrates a perspective view of an exemplary porous mechanism or membrane 3712 for covering a reservoir in the delivery devices of FIG. 8B, in which the porous membrane 3712 includes one or more orifices or ports 3714 for releasing the substance. In an exemplary embodiment, there are three orifices in each reservoir porous membrane, and the orifices are square and each have a length of about 80 μm; however any other suitable number of pores may be used. An exemplary array of delivery devices may include any suitable number of reservoirs including, but not limited to, 2, 3, 4, 5, 6, 7, 8, 9, 10, and more.

FIGS. 8D-8F illustrate a further exemplary embodiment of a wearable delivery device. For example, delivery device 3722 is an exemplary delivery device that can be smaller in size than delivery device 2800 described above. The smaller size of delivery device 3722 can facilitate delivery device 3722 to be used as a part of, or as, a piece of clothing, or an item that can attach to a person's body. Delivery device 3722 can include one or reservoirs similar to reservoirs 2802 described above. As shown in FIG. 8F, delivery device 3722 can include heat transfer element 3720, which can be incorporated into a bracelet 3726, which can fit around the arm or wrist of a user. Heat transfer element 3720 can facilitate a transfer of body heat from the wearer of the clothing to the array 3704, to heat the substance in array 3704.

In an experiment to determine the efficacy of exemplary devices, four (4) arrays of delivery devices were provided along the length of a human arm over a Flame Resistant Army Combat Uniform (FRACU) sleeve worn by the arm. Each array of devices included six (6) reservoirs filled with 20 cubic mm of 10% transfluthrin. FIG. 9 illustrates an experimental setup of a controlled experiment performed to determine the efficacy of the exemplary array in deterring pest bites. In the experimental setup, a closed cage 3802 was provided to accommodate approximately two hundred (200) Aedes aegypti mosquitoes. An arm 3804 of a human subject was positioned within the cage 3802 through a narrow opening that prevents the exit of the mosquitoes. During the control phase of the experiment, the arm 3804 was covered by a Flame Resistant Army Combat Uniform (FRACU) sleeve 3806 and was exposed to the mosquitoes for about 15 minutes per day. During the test phase of the experiment, the arm 3804 was covered by a FRACU sleeve and fitted with four arrays of devices, each array including six reservoirs. The test arm was exposed to the mosquitoes for about 15 minutes per day. At the end of the experiment, the mosquitoes were killed with carbon dioxide, and examined to determine if they contained human blood in order to quantify the number of mosquito bites that had occurred.

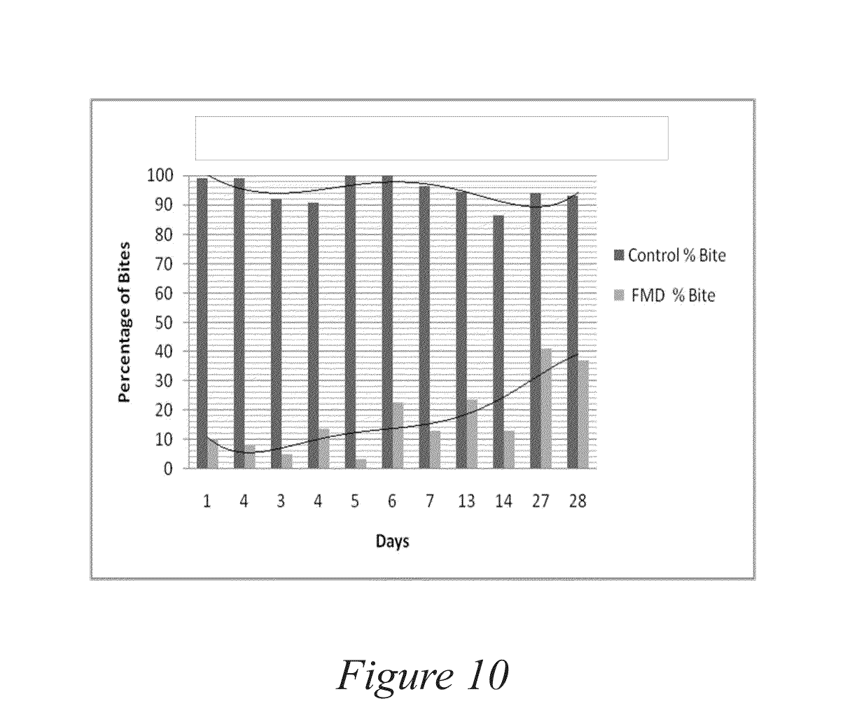

A surprising and unexpected result of the experiment was the discovery that exemplary delivery devices resulted in a significant and marked reduction in the percentage of mosquitoes that bit the arm equipped with an array of six delivery devices, in which each reservoir membrane included three square orifices having a length of about 80 μm, as compared to the percentage of mosquitoes that bit the arm lacking the array of delivery devices. FIG. 10 is a histogram of the percentage of mosquitoes that bit the test arm over 28 days of the experiment. As shown in the histogram, on each day of the experiment, there was a significant and marked reduction in the percentage of mosquitoes that bit the test arm compared to the control arm. On day 1, there was an almost 10-fold decrease in the percentage of mosquitoes that bit the test arm. On day 2, there was approximately a 12-fold decrease. On day 3, there was approximately a 23-fold decrease. On day 4, there was approximately a 6-fold decrease. On day 5, there was approximately a 33-fold decrease. On day 6, there was approximately a 4-fold decrease. On day 7, there was approximately an 8-fold decrease. On day 13, there was approximately a 4-fold decrease. On day 14, there was approximately a 7-fold decrease. On day 27, there was approximately a 2-fold decrease. On day 27, there was approximately a 2-fold decrease. The experimental results show that exemplary delivery devices that release a pesticide are reliably and significantly more effective in reducing the number of mosquito bites than conventional technologies, such as those that use clothing treated with the same pesticide.

The use of exemplary delivery devices resulted in markedly low percentages of mosquitoes that bit the test arm. For example, on day 1, only approximately 10% of the mosquitoes bit the test arm; on day 2, approximately 8% of the mosquitoes bit the test arm; on day 3, approximately 4% of the mosquitoes bit the test arm; on day 4, approximately 14% of the mosquitoes bit the test arm; on day 5, approximately 3% of the mosquitoes bit the test arm; on day 6, approximately 22% of the mosquitoes bit the test arm; on day 7, approximately 13% of the mosquitoes bit the test arm; on day 13, approximately 24% of the mosquitoes bit the test arm; on day 14, approximately 12% of the mosquitoes bit the test arm; on day 27, approximately 41% of the mosquitoes bit the test arm; and on day 28, approximately 37% of the mosquitoes bit the test arm. These low percentages of mosquito bites are a significant improvement over conventional technologies, such as those that use clothing treated with the same pesticide.

Another surprising and unexpected result of the experiment was the discovery that exemplary delivery devices resulted in a sustained reduction in the percentage of mosquitoes that bit the arm equipped with the array of devices, as compared to the percentage of mosquitoes that bit the arm lacking the array of devices. Exemplary delivery devices were able to release the pesticide in a sustained manner over a long period of time, for example, at least 28 days in this experiment. This enabled the reduction of mosquito bites over the entire course of the experiment, i.e. over the 28 days. Even on days 27 and 28—that is upon expiration of a large period of time since the first use—exemplary delivery devices showed significant efficacy in reducing the percentage of mosquito bites. The use of the exemplary delivery devices reduced the percentage of bites, compared to the control case, for a period of four weeks. The exemplary devices reduced the number of bites by approximately 80% over a sustained period of three weeks.

The experimental results showed that exemplary delivery devices provide an effective means for releasing insecticides and in protecting against pests such as mosquitoes. The experimental results showed that three exemplary delivery devices effectively released the pesticide over an area of about 100 square cm, which is equivalent to one exemplary device being sufficient in providing effective protection against mosquitoes per 33 cubic cm of space.

A summary of the experimental results is provided in Table 2.

TABLE 2
Summary of experimental results on efficacy of exemplary delivery
devices in reducing the percentage of mosquitoes that bite an arm

| | | Mos- | | Test | Number | | |
|---|---|---|---|---|---|---|---|
| | | quito | Sample | Popu- | of | % of | |
| Date | Time | Age | Tested | lation | Bites | Bites | BP % |
| 4/12 | 1039 | 11 | Control | 120 | 109 | 90.8 | |
| 4/12 | 106 | 11 | 4 arrays, each | 97 | 70 | 72.2 | 20.6 |
| | | | including 1 | | | | |
| | | | reservoir | | | | |
| 4/13 | 1000 | 12 | Control | 97 | 84 | 86.6 | |
| 4/13 | 1020 | 12 | 4 arrays, each | 98 | 10 | 10.2 | 88.2 |
| | | | including 6 | | | | |
| | | | reservoirs | | | | |
| 4/14 | 712 | 6 | Control | 109 | 108 | 99.1 | |
| 4/14 | 730 | 6 | 4 arrays, each | 129 | 10 | 7.8 | 92.2 |
| | | | including 6 | | | | |
| | | | reservoirs | | | | |
| 4/15 | 723 | 7 | Control | 104 | 103 | 99.0 | |
| 4/15 | 742 | 7 | 4 arrays, each | 123 | 6 | 4.9 | 95.1 |
| | | | including 6 | | | | |
| | | | reservoirs | | | | |
| 4/18 | 1204 | 10 | Control | 102 | 94 | 92.2 | |
| 4/18 | 1223 | 10 | 4 arrays, each | 110 | 15 | 13.6 | 85.2 |
| | | | including 6 | | | | |
| | | | reservoirs | | | | |
| 4/19 | 1147 | 11 | Control | 118 | 107 | 90.7 | |
| 4/19 | 1205 | 11 | 4 arrays, each | 122 | 4 | 3.3 | 96.4 |
| | | | including 6 | | | | |
| | | | reservoirs | | | | |
| 4/20 | 1013 | 6 | Control | 114 | 114 | 100.0 | |
| 4/20 | 1030 | 6 | 4 arrays, each | 120 | 27 | 22.5 | 77.5 |
| | | | including 6 | | | | |
| | | | reservoirs | | | | |
| 4/21 | 123 | 7 | Control | 120 | 120 | 100.0 | |
| 4/21 | 142 | 7 | 4 arrays, each | 105 | 14 | 13.3 | 86.7 |
| | | | including 6 | | | | |
| | | | reservoirs | | | | |
| 4/22 | 819 | 8 | Control | 107 | 103 | 96.3 | |
| 4/22 | 838 | 8 | 4 arrays, each | 122 | 16 | 13.1 | 86.4 |
| | | | including 6 | | | | |
| | | | reservoirs | | | | |
| 4/25 | 113 | 11 | Control | 109 | 103 | 94.5 | |
| 4/25 | 132 | 11 | 4 arrays, each | 110 | 26 | 23.6 | 75.0 |
| | | | including 6 | | | | |
| | | | reservoirs | | | | |
| 4/26 | 204 | 12 | Control | 103 | 89 | 86.4 | |
| 4/26 | 222 | 12 | 4 arrays, each | 124 | 16 | 12.9 | 85.1 |
| | | | including 6 | | | | |
| | | | reservoirs | | | | |
| 5/09 | 120 | 11 | Control | 116 | 109 | 94.0 | |
| 5/09 | 137 | 11 | 4 arrays, each | 120 | 49 | 40.8 | 56.5 |
| | | | including 6 | | | | |
| | | | reservoirs | | | | |
| 5/10 | 1021 | 12 | Control | 115 | 107 | 93.0 | |
| 5/10 | 1038 | 12 | 4 arrays, each | 114 | 42 | 36.8 | 60.4 |
| | | | including 6 | | | | |
| | | | reservoirs | | | | |

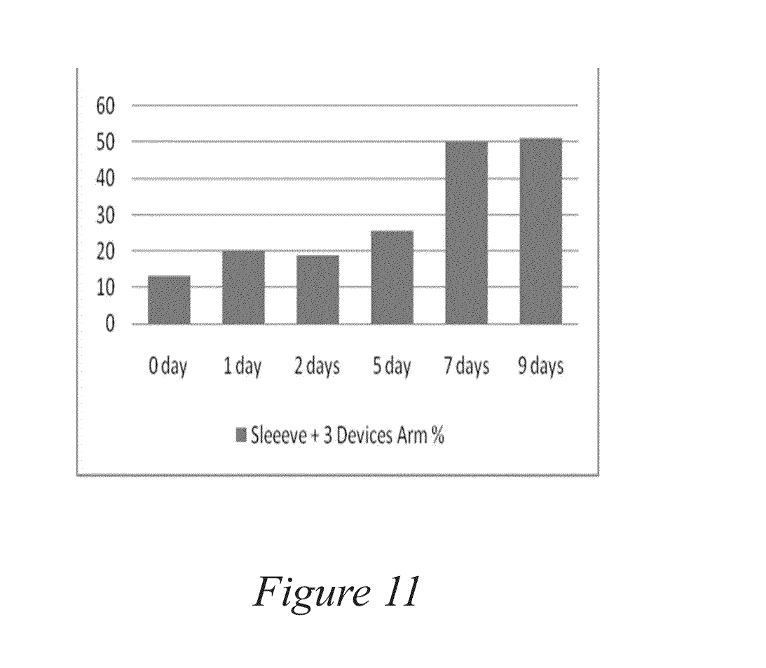

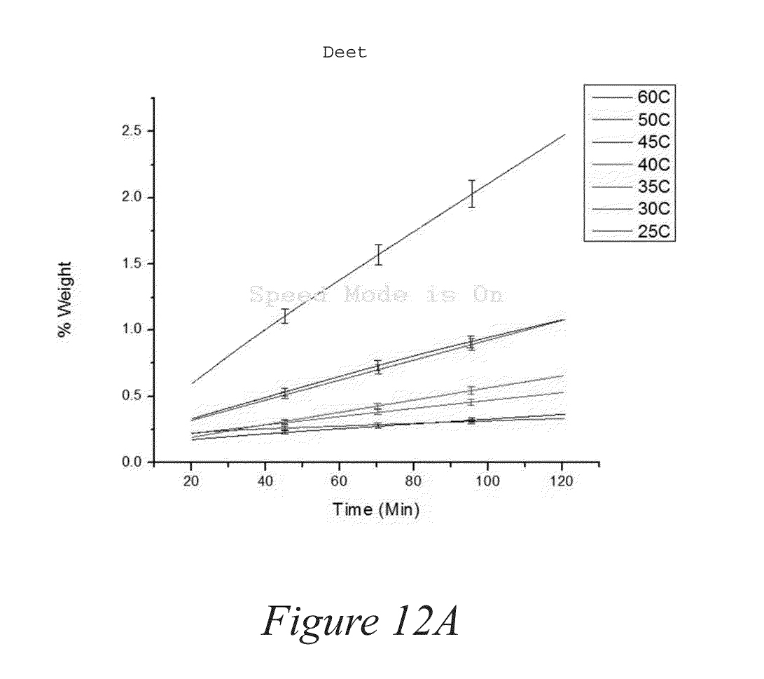

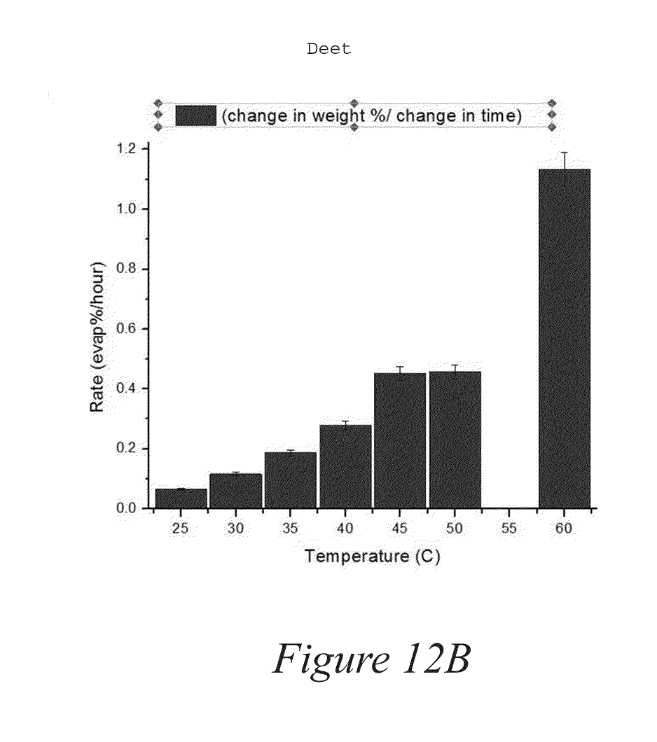

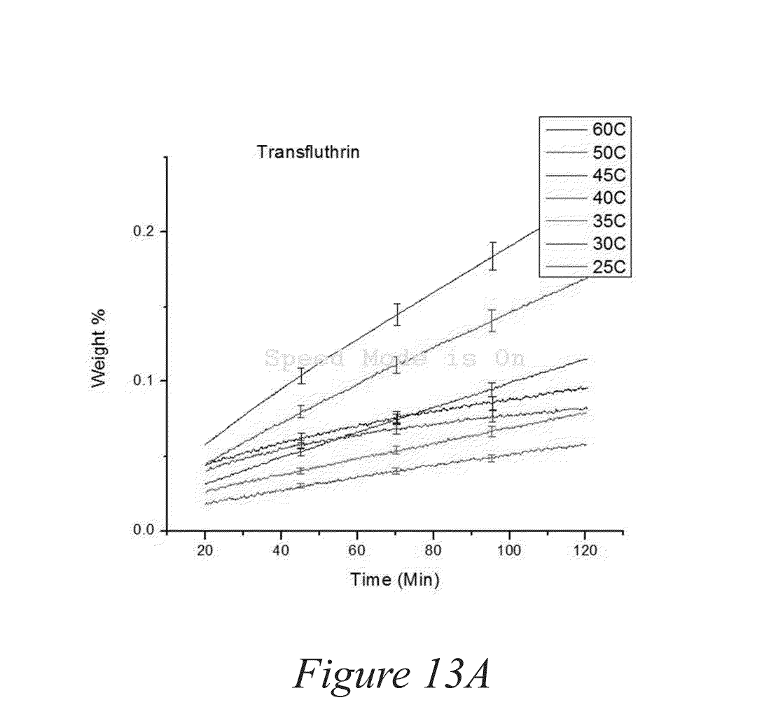

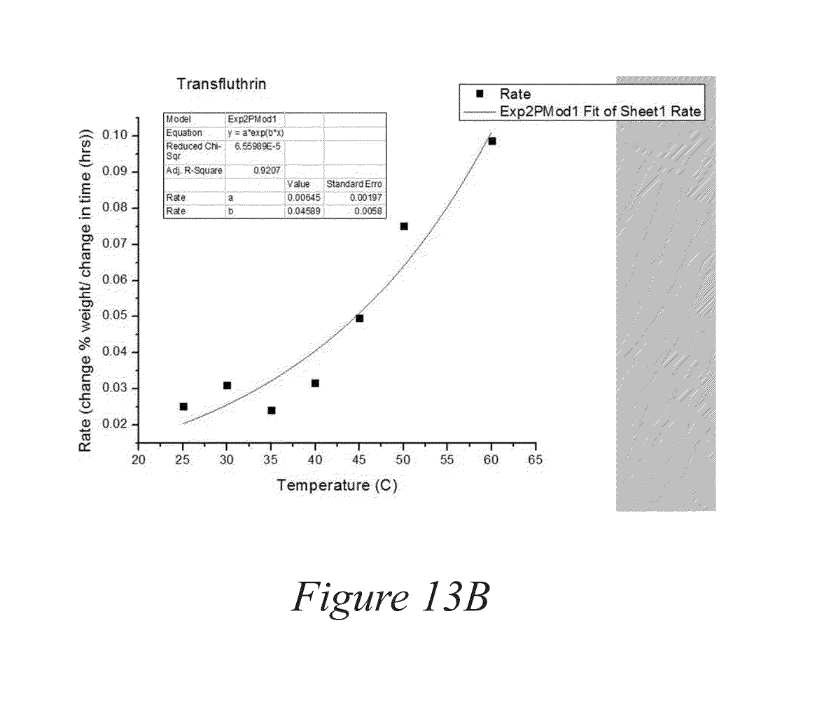

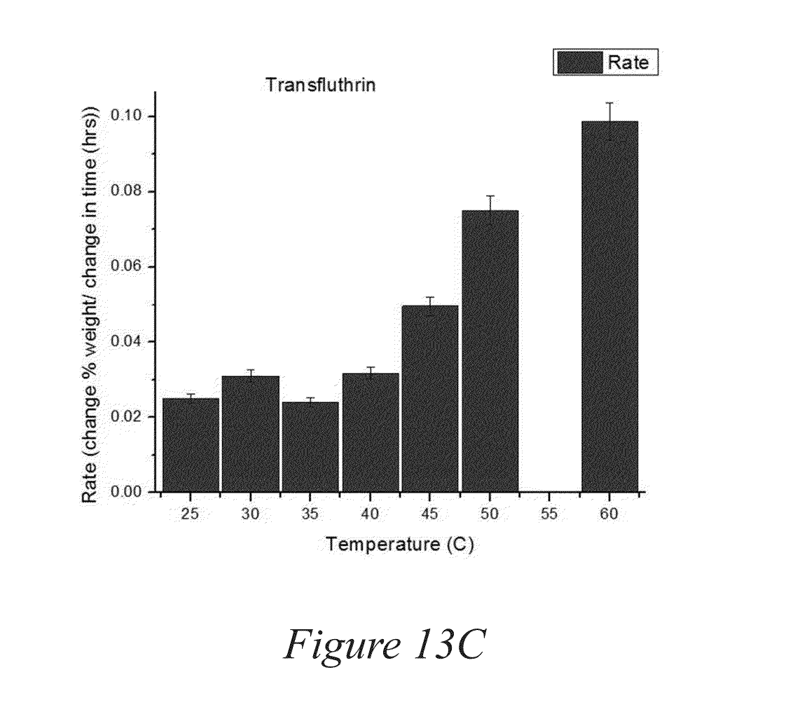

In another experiment, the test arm was provided with three exemplary delivery devices similar to those used in the prior described experiment, and one hundred fifty (150) mosquitoes provided in the cage. The three devices were spaced along the surface of the arm. The other variables of the experiment were the same as the prior described experiment. FIG. 11 is a histogram showing the percentage of mosquito bites of the test arm along the y-axis and the number of days of the experiment along the x-axis.

A summary of the experimental results is provided in Table 3.

TABLE 3
Summary of experimental results on efficacy of exemplary delivery
devices in reducing the percentage of mosquitoes that bite an arm

| | | Mos- | | Test | Number | | |
|---|---|---|---|---|---|---|---|
| | | quito | Sample | Popu- | of | % of | |
| Date | Time | Age | Tested | lation | Bites | Bites | BP % |
| 10/27 | 1315 | 8 | Untreated | 119 | 58 | 48.7 | n/a |
| | | | delivery | | | | |
| | | | device | | | | |
| 10/27 | 1336 | 8 | 1 delivery | 122 | 41 | 33.6 | 31.0 |
| | | | device | | | | |
| 10/27 | 1440 | 8 | 3 delivery | 110 | 24 | 21.8 | 55.2 |
| | | | devices with | | | | |
| | | | 20 mg at cuff | | | | |
| 10/27 | 1500 | 8 | 3 delivery | 114 | 15 | 13.2 | 73.0 |
| | | | devices | | | | |
| | | | spread along | | | | |
| | | | arm | | | | |
| 10/28 | 1441 | 7 | 3 delivery | 98 | 20 | 20.4 | 58.1 |
| | | | devices (after | | | | |
| | | | 24 hrs) | | | | |
| 10/29 | 1415 | 8 | 3 delivery | 96 | 18 | 18.8 | 61.5 |
| | | | devices (after | | | | |
| | | | 48 hrs) | | | | |
| 11/01 | 1255 | 11 | 3 delivery | 113 | 29 | 25.7 | 47.3 |
| | | | devices (after | | | | |
| | | | 5 days) | | | | |
| 11/03 | 1340 | 6 | 3 delivery | 108 | 54 | 50.0 | −2.6 |
| | | | devices (after | | | | |
| | | | 7 days) | | | | |
| 11/05 | 1044 | 8 | 3 delivery | 90 | 46 | 51.1 | −4.9 |
| | | | devices (after | | | | |
| | | | 9 days) | | | | |

One of ordinary skill in the art will recognize that exemplary delivery devices may be used to dispense or deliver any other suitable substances. Certain delivery devices may have one or more reservoirs filled and/or pre-loaded with fragrant substances that are released as scents, fragrances or deodorants into the surrounding air. In some exemplary devices, a single fragrant substance may be provided for release into the air. In other exemplary devices, a plurality of fragrant substances may be provided (for example, in a plurality of reservoirs) for release into the air. The plurality of substances may be released concurrently from an exemplary device, or may be released in a random order or in a predetermined order, for example, the scent of oranges released five minutes after the scent of apples is released.

The exemplary devices may be configured to continuously release the substances. Certain exemplary devices may be pre-programmed and configured to release one or more substances at predetermined times or upon detection of one or more predetermined factors, for example, environmental factors (e.g., a predetermined temperature range, predetermined humidity range), and the like. Certain exemplary devices may be configured to release the substances on demand, for example, upon activation of the devices by a user.

In describing exemplary embodiments, specific terminology is used for the sake of clarity. For purposes of description, each specific term is intended to at least include all technical and functional equivalents that operate in a similar manner to accomplish a similar purpose. Additionally, in some instances where a particular exemplary embodiment includes a plurality of system elements, device components or method steps, those elements, components or steps may be replaced with a single element, component or step Likewise, a single element, component or step may be replaced with a plurality of elements, components or steps that serve the same purpose. Moreover, while exemplary embodiments have been shown and described with references to particular embodiments thereof, those of ordinary skill in the art will understand that various substitutions and alterations in form and detail may be made therein without departing from the scope of the invention. Further still, other aspects, functions and advantages are also within the scope of the invention.

Exemplary flowcharts are provided herein for illustrative purposes and are non-limiting examples of methods. One of ordinary skill in the art will recognize that exemplary methods may include more or fewer steps than those illustrated in the exemplary flowcharts, and that the steps in the exemplary flowcharts may be performed in a different order than the order shown in the illustrative flowcharts.

What is claimed is:

1. A delivery device for sustained release of a substance, the delivery device comprising:
a substrate;
at least one reservoir formed in the substrate for holding the substance; and
at least one porous membrane associated with the reservoir for sustained release of the substance through the pores of the membrane.

2. The delivery device of claim 1, wherein the substance is at least one of a pesticide or a perfume.

3. The delivery device of claim 1, wherein the porous membrane is configured to cover an aperture in the reservoir.

4. The delivery device of claim 1, wherein the substrate is biodegradable.

5. The delivery device of claim 1, further comprising:
a sensor for detecting an environmental characteristic around the delivery device.

6. The delivery device of claim 1, further comprising:
a propulsion mechanism for propelling the substance out of the reservoir.

7. The delivery device of claim 1, further comprising:
a heating mechanism for heating the substance in the reservoir.

8. The delivery device of claim 7, wherein the heating mechanism is at least one of a powered heating element or an exothermic reaction generated by at least one chemical.

9. The delivery device of claim 8, wherein the powered heating element includes at least one metallic element connected to at least one battery.

10. The delivery device of claim 8, further comprising a chamber located below the reservoir for housing the at least one chemical.

11. The delivery device of claim 8, wherein the at least one chemical is at least one of iron oxide, calcium carbonate or calcium carbonate. 12. The delivery device of claim 1, further comprising:
an attachment mechanism for attaching the delivery device to a garment.

13. The delivery device of claim 12, wherein the garment is a bracelet.

14. The delivery device of claim 12, further comprising:
at least one heat transferring arrangement configured to transfer body heat from a person wearing the garment to the substance.

15. The delivery device of claim 1, further comprising:
a self-righting arrangement configured to orient the delivery device in an upward direction when the delivery device is falling.

16. An array of delivery devices for sustained release of a substance, comprising:
two or more delivery devices, each of the two or more delivery devices including:
a substrate;
at least one reservoir formed in the substrate for holding the substance; and
at least one porous membrane associated with the reservoir for sustained release of the substance through the pores of the membrane.

17. The array of claim 16, wherein porous membranes of at least two or more of the delivery devices are oriented in the same direction.

18. The array of claim 16, wherein porous membranes of at least two or more of the delivery devices are oriented in different directions.

19. The array of claim 16, wherein each delivery device further includes at least one protrusion and at least one opening, wherein a protrusion of one delivery device is configured to fit into an opening of a further delivery device to facilitate stacking of the two or more delivery devices.

20. A method for releasing a substance, comprising the steps of:
providing a delivery device including:
a substrate;
at least one reservoir formed in the substrate for holding the substance; and
at least one porous membrane associated with the reservoir for sustained release of the substance through the pores of the membrane;
adding the substance to the at least one reservoir; and
placing the delivery device at a particular location to release the substance.
